(19) United States
(12) Patent Application Publication
(10) Pub. No.: US 2006/0252999 A1
(43) Pub. Date: Nov. 9, 2006

(54) Method and system for wearable vital signs and physiology, activity, and environmental monitoring

(76) Inventors: Richard W. Devaul, Somerville, MA (US); Daniel Barkalow, Somerville, MA (US); John Carlton-Foss, Weston, MA (US); Christopher Elledge, Arlington, MA (US)

Correspondence Address: BERGMAN KUTA LLP, P. O. BOX 400167, CAMBRIDGE, MA, 02140, US

(21) Appl. No.: 11/121,799

(22) Filed: May 3, 2005

(51) Int. Cl. A61B 5/00

(52) U.S. Cl. 600/300; 128/903; 128/920

(57) ABSTRACT

A remote monitoring system includes an on-body network of sensors and at least on analysis device controlled by a hub. The sensors monitor human physiology, activity and environmental conditions. The monitoring system includes a data classifier to take sensor input to determine a condition of the person wearing the remote monitoring system. The remote monitoring system is further able to determine a level of confidence in the determined condition.

### GOVERNMENT RIGHTS

This invention was derived from work partially funded by the Government under contract no. F33615-98-D-6000 from the Air Force Research Laboratory to Sytronics, Inc., and subcontract Sytronics P.O. no. 1173-9014-8001 by Sytronics to AKSI Solutions LLC. The Government retains certain rights in portions of the invention.

### BACKGROUND

Many people, such as soldiers, police, fire fighters, rescue workers, etc., work under hazardous and life-threatening conditions. Many other people are at increased risk of injury or death as the result of a chronic health condition, or complications resulting from the treatment of acute illness, disability, or advancing age. Other people suffer from chronic, or at least sustained, conditions that require long-term monitoring and treatment. People in all of these circumstances may benefit from continuous monitoring, automatic real-time analysis, and proactive reporting of important changes in their health, physiology, activity state, or environmental conditions. Furthermore, those who are responsible for diagnosing, caring for, rescuing, treating, or developing medications for such individuals may also benefit significantly from such monitoring by allowing more timely, less risky, and less expensive interventions. For example, soldiers, fire fighters, rescue workers, and many other first-responders work under hazardous conditions. These individuals could benefit greatly from advance warning of hazardous environmental conditions, fatigue, illness, or other problems. Such information could allow for improved performance, the avoidance of injury or death, and the timely notification of individuals, team members, and rescue workers in the event that unusual hazards are detected or intervention is needed. Furthermore, in situations where intervention resources are limited or rescue is difficult or dangerous, this information could be invaluable for risk management and triage, allowing individuals in the field, team-members, and rescue workers to make better decisions about such matters as the deployment of human resources. By providing individuals, team-members, and rescuers with salient, timely information, everyone involved benefits from improved situation awareness and risk management.

Likewise, for those suffering from acute or chronic illness, or for those who are at elevated risk for illness or injury, the timely detection and automated reporting of life-threatening injury, disease onset, or medical complication could mean the difference between life and death. Even more valuable than the automatic detection of a crisis may be the reporting of danger signs or leading indicators that may allow a crisis to be avoided all together.

Humans respond differently to different conditions. For example, stressors such as heat and dehydration become critical at different levels for different people. Further, a person with heart disease has a different cardiovascular response that a person with heart disease. In short, people respond somewhat differently to stimuli and stressors than other people. An effective monitoring system would take this into account.

Information relevant to attempts to address these problems includes work at the U.S. Army Research Institute of Environmental Medicine (USARIEM), a part of Natick Laboratories of the United States Army. The USARIEM discloses a hand-sized monitor that miniaturizes Bruel and Kjaer instruments for measuring wet bulb and dry bulb temperature that have transformed heat risk assessment. Data from this monitor is translated to an algebraically calculated estimate of risk from heat stress for lowered productivity or work stoppage and heat prostration. This device is not based on any individual's data. That is, the device assumes that all people are the same. The device is a local monitor, lacking the proactive remote notification features.

Another device in the conventional art is the hand-held doctor project of Richard DeVaul and Vadim Gerasimov of the MIT Media Lab. The hand-held doctor includes a device having sensors for temperature, heart beating and breathing to be used to monitor a child's body. The hand-held doctor further includes infra-red connectivity to a robot which performed actions that reflected the measurements. The first and only prototype of the hand-held doctor system included a small personal Internet communicator-based (i.e., PIC-based) computer with analog-to-digital converters and a radio frequency transmitter, three hand-built sensors, a robot with a receiver, and a software program. The sensors included a thermosensor to measure body temperature, a thermistor-based breathing sensor, and an IR reflectance detector to check the pulse.

Also developed at the MIT Media Lab, the “Hoarder Board,” designed by Vadim Gerasimov, had the purpose of collecting large amounts of sensor data. The board can be configured and programmed for a range of data acquisition tasks. For example, the board can record sound with a microphone add-on board or measure electrocardiographic data, breathing, and skin conductivity with a biometric daughter board. The board can use a CompactFlash device to store sensor information, a two-way radio modem or a serial port to communicate to a computer in real time, and a connector to work in a wearable computer network. When combined with a biometric daughter board or multi-sensor board, the system is capable of physiology monitoring or activity monitoring with local (on-device) data storage. The board also supported a simple low-bandwidth point-to-point radio link, and could act as a telemonitor. The board has a small amount of processing power provided by a single PIC microcontroller and a relatively high overhead of managing the radio and sensors.

Further conventional art includes products of BodyMedia Co. of Pittsburgh, Pa. BodyMedia provides wearable health-monitoring systems for a variety of health and fitness applications. The core of the BodyMedia wearable is a sensing, recording, and analysis device worn on the upper arm. This device measures several physiological signals (including heart rate, skin temperature, skin conductivity, and physical activity) and records this information for later analysis or broadcasts it over a short-range wireless link. The BodyMedia wearable is designed to be used in conjunction with a server running the BodyMedia analysis software, which is provided in researcher and end-user configurations, and in an additional configuration that has been customized for health-club use.

Other conventional wearable remote monitoring systems include alert systems that set off an alert when a condition exceeding a selected threshold is detected. One example of such a system is the Personal Alert Safety System (PASS) worn by firefighters.

It remains desirable to have a method and apparatus for wearable monitoring with real-time classification of data.

### SUMMARY

The problems of monitoring individual comfortably, accurately and with the ability to generate notification of hazardous conditions with a level of confidence are solved by the present invention of a wearable monitor including real-time analysis.

Although the wearable component of the Media Lab device (the hand-held doctor) provides physiological telemonitoring capabilities (it streams raw, uninterpreted physiology data over an infrared wireless communications system) it lacks real-time analysis capabilities and accordingly does not provide proactive communications features.

The Hoarder board has a small amount of processing power and accordingly lacks real-time analysis capabilities. For example, the Hoarder board also does not provide proactive communications.

Although the BodyMedia wearable system is capable of real-time telemonitoring and at least some remote real-time analysis, the system continuously captures or wirelessly streams data in real-time to a remote location where analysis can be done.

In contrast, the present inventive technology is specifically designed for the real-time, continuous analysis of data (which may, in some embodiments of the invention, be recorded), and to proactively relay this information and analysis when dangerous or exceptional circumstances are detected. The advances of the present inventive technology include managing power consumption and communications bandwidth.

Further, those conventional systems including an alert system typically operate using simple threshold values which make them somewhat dysfunctional under real world conditions. Whether or not a hazard actually exists is often determinable only by combinations of factors and conditions. Alert systems using simple threshold values often misinterpret the data input. The Personal Alert Safety System (PASS) alarms used by firefighters are a good example of one such dysfunctional alert system. PASS alarms create a considerable nuisance with their false positive responses, and firefighters are therefore inclined to disengage them or ignore them. The problems associated with false positives may in some cases be mitigated by bringing the wearers into the interaction loop by means such as giving them the opportunity to cancel an automatically triggered call for help. This, however, only transfers the burden from one set of individuals (the rescuers) to another (the wearers). While this may reduce the economic cost of false positives it may also place an unacceptable cognitive burden on the wearer.

The present invention relates to the use of body-worn or implanted sensors, microelectronics, embedded processors running statistical analysis and classification techniques, and digital communications networks for the remote monitoring of human physiology, activity, and environmental conditions; including vital-signs monitoring; tracking the progress of a chronic or acute ailment; monitoring exertion; body motions including gait and tremor, and performance; detecting injury or fatigue; detecting environmental conditions such as the buildup of toxic gas or increasing external temperature; the detection of exposure to toxic chemicals, radiation, poisons or biological pathogens; and/or the automated detection, real-time classification, and remote communication of any other important and meaningful change in human physiology, activity, or environmental condition that may require notification, treatment, or intervention.

All of these monitoring, interpretation, and proactive communications applications have at their foundation a combination of sensing, real-time statistical analysis, and wireless communications technology. Furthermore, this technology is packaged in a manner that is as comfortable and non-invasive as possible, and puts little additional physical or cognitive burden on the user. It is robust and reliable, unobtrusive, accurate, and trustworthy. It is as simple as possible to operate, and very difficult to break.

A preferred embodiment of the present invention is a wearable system including one or more small, light-weight electronics/battery/radio packages that are designed to be integrated into the wearer's current uniform, equipment, or clothing. These may be packaged as separate, special-purpose devices, integrated into existing gear (watches, cell phones, boots or equipment harnesses, pagers, hand-held radios, etc.), or incorporated directly into clothing or protective gear.

Sensor Hub

The center of the wearable system is a sensor hub. If the wearable is monolithic, the sensor hub is a package containing all sensors, sensor analysis hardware, an appropriate power source, and an appropriate wireless communications system to proactively contact interested third parties. The sensor hub package also supports whatever wearer-interaction capabilities are required for the application (screen, buttons, microphone/speaker, etc.) For some applications, a distributed, multi-package design is more appropriate. In these cases, there is a distinguished sensor hub responsible for communicating relevant information off-body, but some or all of the sensing, analysis, and interaction is done in separate packages, each of which is connected to the central package through an appropriate personal area network (PAN) technology.

Personal Area Network

For the distributed wearable configuration, the on-body components are tied together through a personal area network. This network can range from an ad-hoc collection of sensor-specific wired or wireless connections to a single homogeneous wired or wireless network capable of supporting more general-purpose digital communications. For example, a particular wearable application may require sensors or electrodes to be placed against the wearer's skin, woven into a garment, or otherwise displaced from the sensor hub's package. In these cases, the sensors, particularly if they are simple analog sensors, are tied to the sensor hub through dedicated wired connections. In another application, for power consumption or standoff detection reasons, several digital sensing or interaction components are tied together with an on-body wired digital personal area network. In other cases, human factors or other usability constraints may make wired connections between some on-body components infeasible; in these cases, an embodiment of the present invention includes a wireless digital personal area network (RF, near-field, IR, etc.) used to tie some or all of the sensing or interaction modules to the sensor hub. Finally, further alternative embodiments of the present invention combine all three of these personal area networking strategies. In the cases where a wireless personal area network is used, all on-body modules participating in the network have an appropriate network transceiver and power source.

Sensor/Analysis Packages

In the case of a distributed, multi-package sensor design, separate packages containing sensors and sensor analysis hardware are distributed about the body as appropriate for the application and usage model. In some embodiments, these packages are analog sensors or electrodes, in which case the “package” is composed of the sensor or contact itself with any necessary protective packaging, appropriately positioned on the wearer's body or incorporated into clothing. In other embodiments, the sensor is a self-powered device with a special-purpose wireless network. In these cases the sensor package includes not only the sensor, but an appropriate transceiver, which in most cases will require a separate power supply. There are completely passive wireless sensors and radio frequency identification (RFID) systems that do not require a power supply, but instead are “powered” through the communications link. In order to conserve power and personal area network bandwidth, some versions of the inventive art will have sensor/analysis packages that combine real-time analysis hardware with the sensor in single package. This version is particularly appropriate for wireless personal area networks in which the cost-per-bit of transmitting data is significantly higher than the cost-per-bit of processing and analyzing sensor data, or in which the available wireless personal area network (WPAN) bandwidth is low. By shifting some of the processing of sensor data away from the sensor hub, lower-bandwidth “summary” or analysis data rather than raw sensor data is sent over the WPAN, thus conserving power and bandwidth.

Wearer Interaction Packages

Some embodiments include user interaction. One or more dedicated user interaction packages are thus included as part of the wearable system to improve usability. Such embodiments may include components as a screen, buttons, microphone, speaker, vibrating motor with the sensor hub or some other sensing/analysis package with an appropriately capable PAN to link it with other parts of the system. For example, in one embodiment, a display is integrated into eyeglasses, safety glasses, or an existing body-worn equipment monitor. Likewise, in another embodiment, an audio alert or interaction system is incorporated into a currently worn body-worn audio communications stem, such as a cell-phone or two-way radio. Other components and arrangements for wearer interaction are possible within the scope of the present invention. The present invention is not limited to those listed here. For example, wearer interaction can also be accomplished by writing new software or firmware modules to enable existing devices to operate with the wearable of the present invention in novel ways. Such devices include cell phones, PDAs, or other currently worn gear that support a wired or wireless communications link with the wearable sensor hub.

Packaging Considerations

One embodiment of the present invention combines a “hard” sensor hub module packaged in an ABS plastic enclosure, and one or more “soft” physiology sensing components that are in direct contact with the skin. Extra care and consideration is taken with these “soft” sensor packages that interact directly with the body. The compatibility of these sensors and their packaging is considered in view of the wearer's activities and other gear and in view of the level of distraction to the user. Improvements in the wearability are achieved when allowable and feasible by minimizing the number of “soft” sensor packages required, and by weaving sensors directly into the fabric of an undershirt, for example, or other existing clothing component.

It is important that the technology described herein is intended for long-term use, and that there is a large difference between designing for short-term wearability and long-term wearability. Many design choices that are acceptable for short-term wearability (and are found in existing biomedical sensing devices) are not acceptable for longer-term use. One example is the temporary use of adhesive electrodes for electrocardiogram (ECG) or other bioelectrical measurement are acceptable to users, but are not well tolerated for longer-term use, such as envisioned by the technology described here. For long-term wearability, adhesive connections to the skin, prolonged contact with nickel steel or other toxic or allergenic materials, and numerous other potentially slightly irritating or uncomfortable materials or configurations are preferably avoided. Another example of a configuration preferable avoided is the temporary use of a highly constraining and somewhat rigidified under-shirt that holds sensors close to the body at the cost of distraction and the inability to move normally. Instead, as discussed above, sensors are ideally woven into normal attire.

The size, weight, and positioning of the “hard” components is a consideration for wearability and usability. Reducing size and weight as much as possible is important, but robustness and compatibility with an appropriate range of activities and existing gear is also important. Positioning hard components on the body is an important factor effecting comfort, especially for wearers who are otherwise encumbered. Wired connections on the body and the mechanical connections associated with them present certain reliability and robustness challenges. They also present challenges in wearability and usability. In applications using the technology described herein, various embodiments include strain relief to protect the cables and wired connections. Frequently made or broken mechanical connections are designed for extreme durability. At the same time, heavy or bulky connectors—which may be required for applications involving gloved users—are selected to minimize the impact on wearability. For these reasons, it is desirable to minimize the number of wired connections and mechanical interfaces for body-worn applications.

The present invention together with the above and other advantages may best be understood from the following detailed description of the embodiments of the invention illustrated in the drawings, wherein:

### DRAWINGS

FIG. 1 is a picture of a chest strap according to principles of the invention;

FIG. 2 is a picture of a chest strap including wires to a hub according to principles of the invention;

FIG. 3 is a block diagram of a first configuration of the hub and sensor placement on a representative human figure according to principles of the invention;

FIG. 4 is a block diagram of a second configuration of the hub and sensor placement on a representative human figure according to principles of the invention;

FIG. 5 is a block diagram of a hub and sensor network according to principles of the invention;

FIG. 6A is a schematic diagram of a first portion of a first hub according to principles of the invention;

FIG. 6B is a schematic diagram of a second portion of the first hub according to principles of the invention;

FIG. 6C is a schematic diagram of a third portion of the first hub according to principles of the invention;

FIG. 6D is a schematic diagram of a fourth portion of the first hub according to principles of the invention;

FIG. 7A is a schematic diagram of a first portion of a second hub according to principles of the invention;

FIG. 7B is a schematic diagram of a second portion of the second hub according to principles of the invention;

FIG. 7C is a schematic diagram of a third portion of the second hub according to principles of the invention;

FIG. 7D is a schematic diagram of a fourth portion of the second hub according to principles of the invention;

FIG. 8 is a flow chart of the statistical classification process according to principles of the invention; and

FIG. 9 is a flow chart of the process of the classifier module according to one embodiment of the invention.

### DESCRIPTION

A remote monitoring system includes a wearable configuration of sensors and data analysis devices and further includes data models for interpretation of the data collected by the sensors. The sensors monitor human physiology, activity and environmental conditions. In one embodiment, the data analysis devices use the data models to determine whether hazardous conditions exist. In other embodiments, features are derived by bandpass filtering, signal processing operations, or other analytics. Some embodiments provide useful displays to the user, where the displays are based on algorithms operating on and displaying raw data alone and combined with derivative data. In another embodiment, a communications system included in the remote monitoring system sends an alarm when the remote monitoring system detects a hazardous condition.

All of these monitoring, interpretation, and proactive communications applications have at their foundation a combination of sensing, real-time statistical analysis, and wireless communications technology. Furthermore, this technology is packaged in a manner that is as comfortable and non-invasive as possible, and puts little additional physical or cognitive burden on the user. It is robust and reliable, unobtrusive, accurate, and trustworthy. It is as simple as possible to operate, and difficult to break. A feature of the system described here is the proactive, robust notification capability provided by the combination of sensing, real-time statistical analysis, and proactive communications. This capability makes it possible to automatically and reliably notify relevant third parties (care-givers, rescuers, team-members, etc.) in the event of emergency or danger.

The body-worn, implanted, and mobile components of the system (hereafter “the wearable”) are highly reliable with long battery (or other mobile power-source, e.g. fuel cell) life, so that both the individual being monitored and those who may be required to intervene can rely on its continued operation over a sufficiently long period of time without the constant concern of power failure. To achieve this, an appropriate power source is selected and the electronics are engineered for low power consumption, particularly for processing and communications. Effective low-power engineering involves careful selection of electronic components and fine-grained power management so that particular subsystems (such as a communications radio, microprocessor, etc.) may be put into a standby mode in which the power consumption is reduced to an absolute minimum, and then awakened when needed.

Human Factors

The human factors of the wearable—both cognitive and physical—are important to the overall usefulness of the system. From the cognitive standpoint the wearable is very simple to use, with as many functions as possible automated, so that the wearer can attend to other tasks with minimal cognitive burden imposed by the device. To the extent that the wearable interacts with the user, the interactions are carefully designed to minimize the frequency, duration, and complexity of the interactions. The physical human factors of the wearable are also important; the wearable's physical package is as small and light as possible, and is carefully positioned and integrated with other body-worn (or implanted) elements so that it will not encumber the user, interfere with other tasks, or cause physical discomfort. Sensors, in particular physiological sensors, are carefully selected and placed for measurement suitability, compatibility with physical activity, and to minimize the physical discomfort of the wearer. Weight and size are important design criteria, requiring both miniaturization of electronics and careful low-power design, since power consumption translates directly into battery (or other mobile power source) weight.

Sensing

Not all locations on the human body are equal with regard to the location of physiological sensors, and in many cases it may be desirable to embed sensors or other components of the system in clothing, shoes, protective gear, watches, prosthetics, etc. Wired connections among distributed on-body wearable components are, at times, infeasible due to human factors or usage constraints, and in such cases a suitable wireless personal-area network is integrated that meets the bandwidth, latency, reliability, and power-consumption requirements of the application. Likewise, a suitable local- or wide-area wireless networking technology has been chosen so that the wearable components of the system may communicate with care givers, rescue workers, team members, or other interested parties.

In many cases, a plurality of sensors are appropriate to measure a signal of interest. In some cases no appropriate single sensor exists. For example, there is no single sensor that can measure mood. In others, constraints of the body-worn application make such sensing impractical due to ergonomic considerations or motion artifacts arising from the ambulatory setting. For example, measuring ECG traditionally requires adhesive electrodes, which are uncomfortable when worn over an extended period. Core body temperature is most reliably sensed by inserting probes into body cavities, which is generally not comfortable under any circumstances. Those skilled in the art will recognize that many additional examples could be identified. In some cases these problems can be mitigated through improved sensor technology (e.g. replacing adhesive electrodes with clothing-integrated fabric electrodes for ECG, or the use of a consumable “temperature pill” for core-body temperature measurement). In other cases, however, a constellation of sensors is applicable. The constellation of sensors parameterize a signal space in which the signal of interests is embedded, and then use appropriate signal processing and modeling techniques to extract the signal of interest.

In some embodiments, the constellation of sensors measure a collection of signals that span a higher-dimensional measurement space in which the lower-dimensional signal of interest is embedded. In these alternative embodiments, the lower-dimensional signal of interest is extracted from the higher-dimensional measurement space by a function whose domain is the higher-dimensional measurement space and whose range is the lower-dimensional measurement space of interest. This function involves, for example, a sequence of operations which transform the representation of the original measurement space. The operations further include projecting the higher-dimensional space to a lower-dimensional manifold, partitioning the original or projected space into regions of interest, and performing statistical comparisons between observed data and previously constructed models.

Automated Real-Time Interpretation of Sensor Signals

Throughout this discussion the general term “model” or “model/classifier” is used herein to describe any type of signal processing or analysis, statistical modeling, regression, classification technique, or other form of automated real-time signal interpretation. Even in situations where the signal of interest is measurable in a straightforward manner that does not burden or discomfort the user, the proper interpretation of this signal may require knowledge of other signals and a the wearer's personal history. For example, it is relatively straightforward to measure heart rate in an ambulatory setting, and increases in heart rate are often clinically meaningful. Simply knowing that the wearer's heart rate is increasing is generally not sufficient to understand the significance of this information. With the addition of information about the wearer's activity state (which can be extracted from the analysis of accelerometer signals) it is possible to distinguish an increase in heart-rate resulting from increased physical activity from one that is largely the result of emotional state, such as the onset of an anxiety attack. Likewise, the cardiovascular response of a fit individual will differ substantially from that of an unfit person. Thus, even for interpreting a relatively straightforward physiological signal such as heart rate, proper interpretation may require additional sensor information as well as additional information about the wearer.

Noise and Uncertainty

Just as measured signals typically contain noise, interpretation typically involves uncertainty. There is a great deal of difference between saying “it is going to rain” and “there is a 35% chance of rain.” Likewise, there is a large difference between an automated interpretation with high confidence and one with low confidence. One source of uncertainty in the interpretation of sensor signals is noise in measurement. Measurement typically involves some degree of noise, and the amount of noise present varies depending on circumstances. For example, many physiological sensors are prone to motion artifacts, and in such cases the amount of noise in the signal is strongly correlated with the amount of motion. Another source of uncertainty lies in the limitations of what can be sensed and modeled—not all relevant parameters can be measured or even known for some important conditions. For example, after decades of research and modeling, the US Army recently discovered when trainees died of hypothermia in a Florida swamp that there was greater variation among various individuals' thermoregulatory capacities than had been previously believed.

In general, models capable of working with and expressing uncertainty are preferable to those which are not. Further, regardless of whether the sensing task is simple or complex, all sensor measurements are a combination of signal and noise, and appropriate analysis techniques takes this into account. Although linear regression, thresholding or other simple modeling and classification techniques may be appropriate for some applications, better results can almost always be obtained through the application of more principled statistical modeling techniques that explicitly take uncertainty into account. This is particularly important for the automated classification of conditions, events, or situations for which there is a high cost for both false-positive and false-negative classification. For example, the failure of a system designed to detect life-threatening injury, cardiac fibrillation, etc. may be life-threatening in the case of a false negative, but expensive and ultimately self-defeating if false positives are common. The Personal Alert Safety System (PASS) alarms presently used by firefighters are a good example of one such dysfunctional alert system because they create a considerable nuisance with their false positive responses, and firefighters are therefore inclined to disengage them or ignore them. The problems associated with false positives may in some cases be mitigated by bringing the wearers into the interaction loop by means such as giving them the opportunity to cancel an automatically triggered call for help. This, however, only transfers the burden from one set of individuals (the rescuers) to another (the wearers). While this may reduce the economic cost of false positives it may also place an unacceptable cognitive burden on the wearer.

Statistical Classification Process

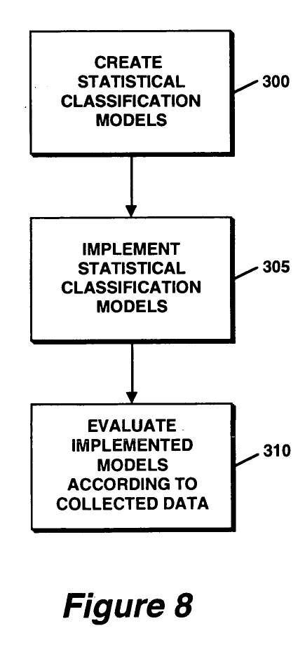

FIG. 8 is a flow chart of a statistical classification process according to principles of the invention. Statistical classification is the process by which measured sensor data is transformed into probabilities for a set of discrete classes of interest through the application of statistical classification techniques. The application of the process summarized here to the problem of wearable telemonitoring systems is one of the key innovations embodied in the inventive system. At step 300, an appropriate set of statistical classification models is created (hereafter to be called “model creation”). At step 305, the statistical classification models resulting from the model creation step are implemented on the wearable such that they can be evaluated in real-time using on-body computational resources (“model implementation”). At step 310, the wearable telemonitor system evaluates these models in real-time using live sensor data, the results of which may trigger communications with remote third parties, cause delivery of status information to the wearer, or otherwise play an important role in the behavior of the wearable telemonitor system. This is the “model evaluation” step.

Model Creation

In general, model creation (step 300) is done once for each class of problem or individual user. In alternative embodiments of the invention, the model is continually refined as the models are used (referred to as “on-line learning”). Unless on-line learning is needed, the model creation process can be done off-line, using powerful desktop or server computers. The goal of the model creation process described here is to create statistical classification models that can be evaluated in real-time using only on-body resources.

Model creation starts with data gathering. In one embodiment of the invention, data is gathered through body-worn sensor data. In general, this data is “labeled” so that what the data represents is known. In some embodiments of the invention, there are two data classes, such as “normal heart activity” and “abnormal heart activity.” Actual example data from both classes is gathered, although there are situations where simulated data may be used if the acquisition of real data is too difficult, costly, or poses some ethical or logistical challenges. From analysis of this representative data, appropriate modeling features are chosen to be used by the model Features are derived measurements computed from the “raw” sensor data. For example, derived measurements in one embodiment are created by computing the differential forward Fourier transform (DFFT) or power spectrum from a short-time windowed sequence of data. Features may also be derived by bandpass filtering, signal integration or differentiation, computing the response of filterbanks or matched filters or other signal processing operations. A “trial feature” is a trial operation which is used to test possible model correlations. The analysis process typically includes the computation of several trial features in order to arrive at a final model feature. After features are chosen, an appropriate model type and structure is chosen. Finally, the parameters for the specific model type, structure, and representative data are estimated from the representative data.

In a first example of an application of the present invention, the sensors are used to measure core body temperature and the data model is the likelihood of morbidity due to heat injury. In this example, the collected data can be analyzed directly according to the morbidity model in order to make conclusions about the severity of the injury.

A second example application of the present invention is a cardiac fitness meter using the cardiac interbeat interval (IBI) at rest to determine cardiac fitness of a subject. A system measuring the duration between heart beats is used to determine the IBI. In order to validate this fitness meter, it is examined against an established, widely recognized fitness assessment system such as a cardiac stress test on a treadmill. An appropriately representative study population is selected which can be done using known techniques in experimentation and statistics. Several minutes of IBI data for each subject at rest is then recorded which results in, for example, two hundred numbers. Then, the subjects are evaluated using the treadmill stress test to establish which subjects are “fit” and which are “unfit,” thus creating model labels. In this example, the “labels” are a continuum, but data cut-offs can be established for analysis purposes. One example of a data cutoff in this instance is the Army minimum fitness standard. Thus, for each subject, the trial feature is computed from the measured interval data. The trial feature (i.e., the IBI variance) is then plotted against the labels, “fit” and “unfit.” An effective fitness meter results in a clear correlation between a higher IBI variance and the “fit” label.

The above examples are simplified, however, the examples demonstrate the point that trial features can be used to construct models to be used with high confidence when using complex, high-dimensional data showing large variations over time or including noise or uncertainty.

Model Implementation

The results of the model creation step (step 300) are: (1) the process for calculating model features, (2) the structure and type of the model, and (3) the model parameters themselves. These three elements specify the statistical classifier. Implementing a model evaluation system (step 305) that is capable of evaluating the statistical classifier in real-time using on-body resources is technically challenging. Feature calculation and model class posterior calculation (i.e., calculating the likelihood that an observed feature, or set of features, is modelable by a particular model class) can be computationally intensive. Although it is often possible to do these calculations using very basic computing resources such as inexpensive microcontrollers, doing so requires the careful selection of appropriate computational resources as well as highly optimized software implementations. A component of this is choosing appropriate algorithms and then implementing them using optimized fixed-point arithmetic. For example, the preferred embodiment includes a very fast algorithm for calculating the Fast Fourier Transform of the sensor data using fixed-point arithmetic rather than floating point arithmetic, because a floating point algorithm would be too slow on a microcontroller.

Model Evaluation

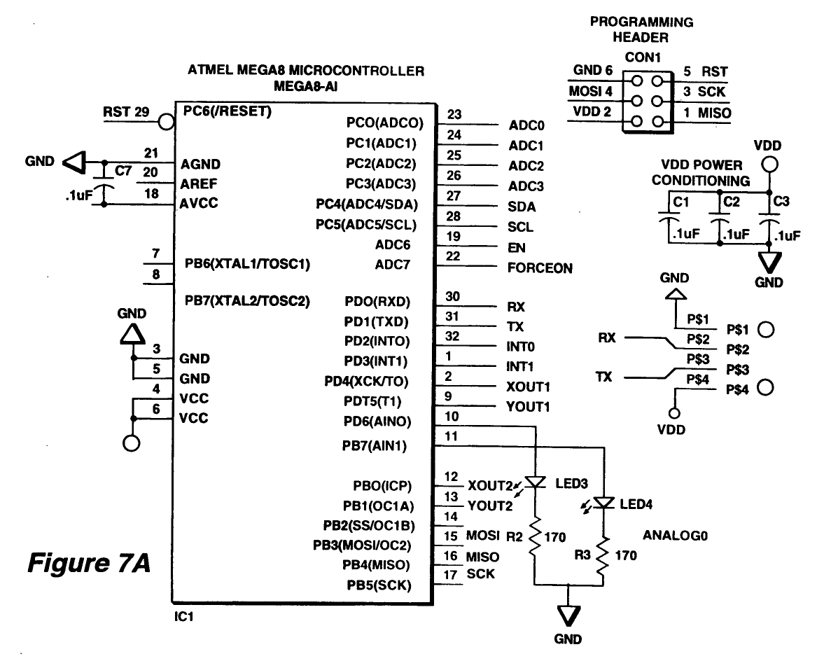

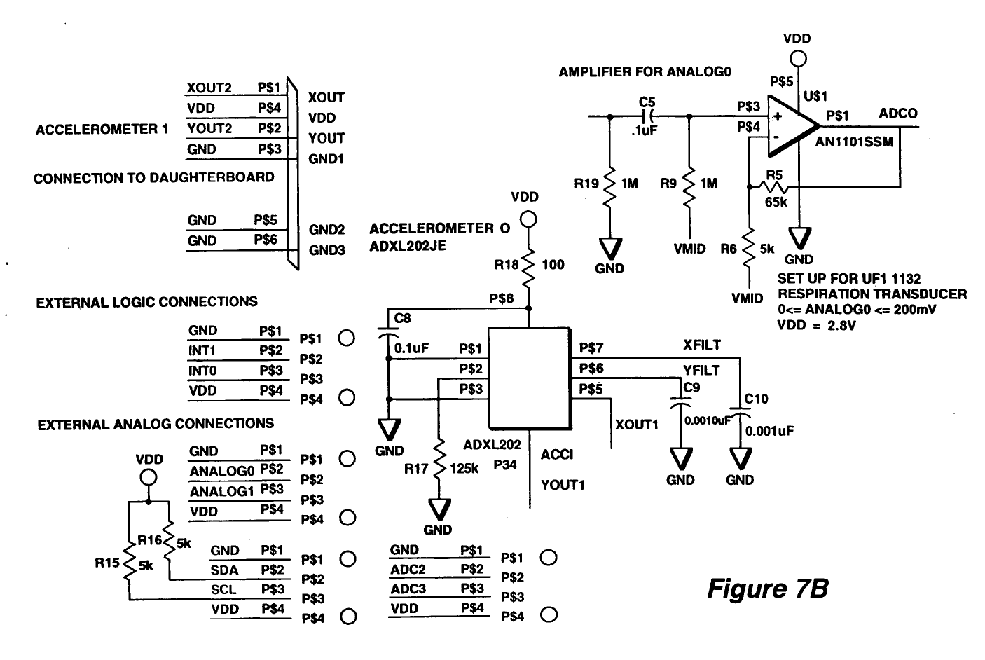

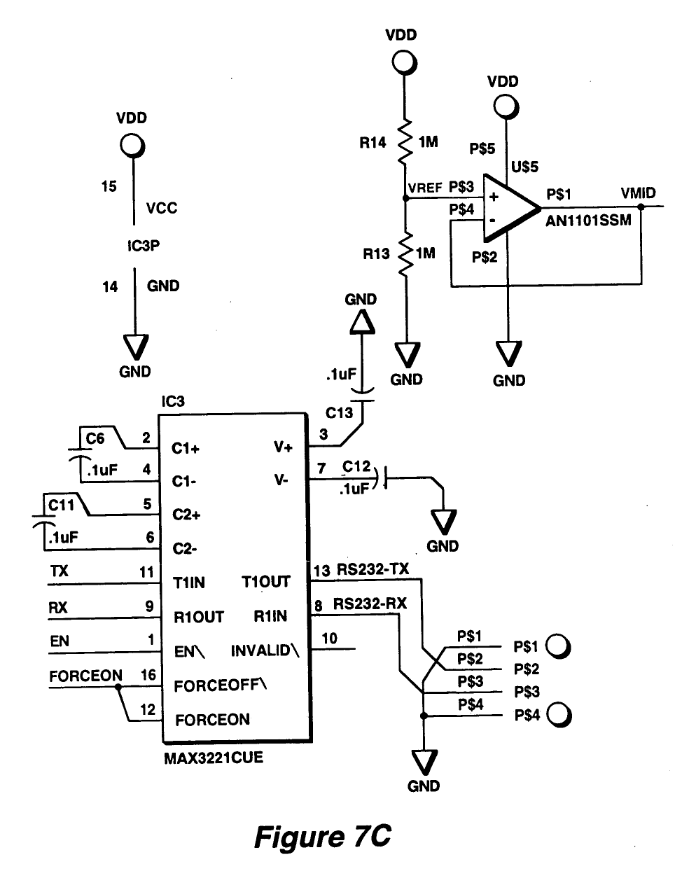

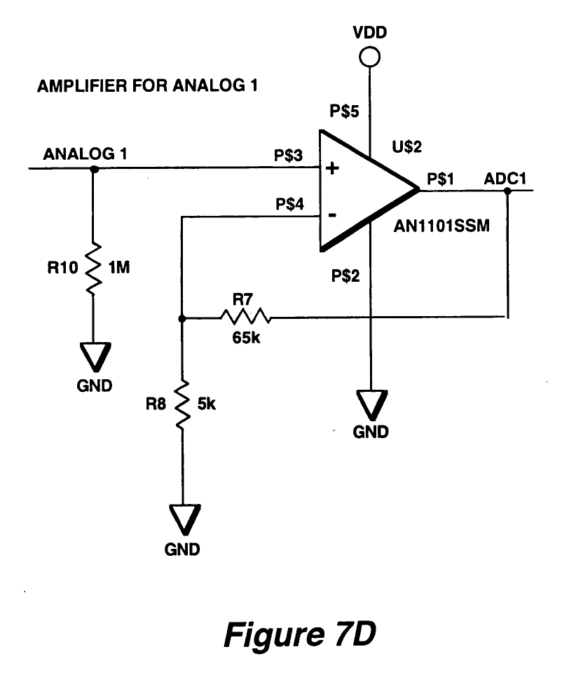

The results of model creation and implementation are a system capable of classifying “live” sensor data in real-time using on-body resources. The step of classification (step 310) entails real time comparison of the features calculated from a data stream to the parameters of the model. This matching using Bayesian statistics identifies the “activity” with which the data stream best matches and yields a statistical estimate of the confidence with which the match can be made. The results of this classification process drive the proactive communications features of the wearable and may otherwise complement information acquired from the wearer, from the wearer's profile or history, and from the network in driving application behavior. An example of model evaluation is described below with regard to FIG. 7A and FIG. 7B.

Distributed vs. Monolithic Wearable Signal Interpretation Architecture—Bandwidth and Power Consumption

The wearable provides sufficient processing power to implement whatever modeling or classification system is necessary for the application. This processing power is provided by local, on-body computing resources, without depending on external computation servers. Modern microcontrollers and low-power embedded processors, combined with low-power programmable digital signal processors (DSPs) or DSP-like field programmable gate arrays most on-body applications. Applications which require distributed on-body sensing may also require on-body distributed computation. Accordingly, in those embodiments with distributed on-body sensing, power at the one or more computational centers on the body and personal area network bandwidth consumption are reduced by performing as much signal processing and modeling as possible in the same package as the sensor. This is particularly important in higher-bandwidth distributed sensing applications (such as distributed wearable systems that employ computer vision systems or speech recognition) in which the raw signal bandwidth may strain the capabilities of the personal area network. In addition, even low-bandwidth distributed sensing applications may benefit from distributed processing since the power cost of wireless communications is almost always higher than computation in modern hardware.

Having the capability to process information on-body is supplemented by the ability to send either the products of the analysis or the original raw data, optionally mediated by the results of on-body analysis, to other locations for further analysis or interpretation of data at a location remote from the body. Indeed, the capability to relay raw sensor signals (be they physiological data, environmental conditions, audio or video, etc.) to remote team members, care givers, or rescuers may be important to the planning and execution of an appropriate intervention. As such, the distributed processing model need not be confined to on-body resources, as the wearable supports a local- or wide-area wireless networking capability in order to be able to communicate with other team members, care givers, rescuers, etc. Such communications are expensive in terms of power consumption, and are generally not preferable for routine operation. If, however, the local- or wide-area communications system is being used for other purposes (such as to call for help, or to provide a “back haul” voice communications channel, etc.) this channel can be important to push data out to “heavy weight” processing resources such as remote computer servers. These servers can be used to provide more sophisticated analysis to the remote team or caregivers. They can also be used to provide additional analysis or interaction capabilities to the wearer (such as a speech-based interface), or to allow for real-time adaptation or modification of the on-body modeling or classification system, including firmware updates and the fine-tuning of model parameters. Those skilled in the art will recognize that the precise computational functionality that is performed, and which of it is performed on the body versus remotely will evolve over the years as microcontrollers become smaller, more powerful and less expensive, and as the applications evolve in purpose and implementation.

Reconfigurable Wearable Signal Interpretation Hardware

Since a single set of sensors can potentially be used for many applications, and because models may be improved over time or tailored to the needs of specific individuals (or even be continuously improved through on-line learning techniques) it is important that the signal processing and interpretation hardware be adaptable. In the preferred embodiment, it is to alter model/classifier parameters, change the model structure or type, or add additional models to be evaluated by updating the wearable's software or firmware, without the need to modify or replace hardware. This is accomplished through the use of self-reprogrammable microcontrollers or conventional embedded/mobile processors (the Intel XScale is an example of one such processor). Alternative embodiments use high-performance reconfigurable signal processing hardware for some or all of the computation, such as programmable DSPs or FPGAs.

Human Machine Interaction

Any explicit interaction demands that the wearable imposes on the wearer will typically translate directly into increased cognitive load and likely decreased task performance. This effect has been documented prior to the development of wearable computers in the form of competing tasks experiments in cognitive psychology. As a result of this phenomenon, it is important to design the human-machine interaction system of the wearable to minimize the frequency, duration, and complexity of these demands. Donald Norman's “Seven Stages of Action” provide a useful framework in which to begin to analyze interaction demands. The seven stages of action are: 1. Forming the goal; 2. Forming the intention; 3. Specifying the action; 4. Executing the action; 5. Perceiving the state of the world; 6. Interpreting the state of the world; and 7. Evaluating the outcome. The Design of Everyday Things, Donald A. Norman, Currency-Doubleday, New York, 1988, pp. 46-48. In particular interactions are carefully designed to minimize Norman's gulfs of evaluation and execution. id., pp. 49-52.

In many cases needed information gathered through explicit interaction with the user can be replaced with information gathered from the automated interpretation of sensor data, augmented with previously stored information and information available through wireless networks. For example, the wearer need not provide location information to rescuers because the information is already available through technologies built into some of the alternative embodiments of the inventive system: a GPS receiver, a dead reckoning system, an RF signal map, or other automated source, taken individually or in some combination. Using information acquired from other sources to reduce the need for explicit user interaction is an important part of mitigating the cognitive demands imposed by the wearable on the wearer, but does not address the entire problem. Interactions that deliver information to the wearer may interfere with other tasks, even when no explicit input is required. Making such information easily understood—reducing Norman's “gulf of evaluation”—is important for reducing the cognitive demands of such interactions. Presenting the wearer with stimuli that require a decision typically interferes with other decision-making tasks. As a result, in the disclosed art any wearable interactions are designed to minimize the presentation of stimuli that require that the wearer make a decision. For example, it would be unreasonable to ask of an airman to remember to turn on his life signs device when he was also involved with making decisions about escaping from a life-threatening situation. Thus, when the device is donned prior to a mission and used with sensors and algorithms to determine whether an airman is alive or dead, it has sufficient battery storage so that it is automatically on and stays on until the airman returns to friendly territory. There is no decision required by the airman to turn it on.

Compatibility with Existing Procedures, Networks, and Equipment

The wearable application is designed for the greatest possible compatibility with existing procedures, activities, and gear used by the wearer. This is important both for reducing the additional training required for effective use of the wearable and to decrease the complications, inconvenience, and expense of adopting the wearable technology. For military and industrial applications this means that the wearable has been designed to function with standard radio gear and networks, standard or existing communications protocols, normal emergency procedures, etc. By leveraging standard body-worn elements such as hand-held radios for long-range communications or personal digital assistants (PDAs) for user interaction, the overall weight, bulk, and complexity of the wearable system is reduced as well.

For civilian biomedical applications, this means that the wearable is designed as much as possible to be unobtrusive, to be compatible with the widest range of street clothing and routine user activities, and to work with (or replace) conventional body-worn devices such as cell phones, PDAs, etc.

### Example Embodiments

Below are described example embodiments of the inventive art constituting the hub, including a variety of alternative embodiments constituting the hub with sensors, peripherals and communications. One embodiment contains its own radio with a range of about 50-100 yards. Another embodiment ties to an electronic device that provides communications to third parties. In another alternative embodiment, a life signs monitor for military personnel uses one of these hubs with sensors to measure heart rate, breathing pattern, GPS (global positioning system), and a three-dimensional accelerometer to measure motion, with selective data sent on demand to an authorize receiver. In another alternative embodiment, a Parkinson's monitor to measure dyskinesia and gait as a means to estimate the need for medication, uses one of the two same hubs, plus accelerometers placed on selected extremities for a period varying from 1 hour to 24 or more hours, with data stored in flash memory or streamed to a separate computer. Still further alternative embodiments employ other combinations of sensors. Those skilled in the art will recognize that the inventive art will support many variations of these same hub, sensor, communications, and linkage configuration for varying purposes. For example, a monitor employing a plurality of sensors can determine a degree of progression of Parkinson's disease or other neurological condition such as stroke or brain lesion that effects for example gait or motion of a patient. Another example monitor according to principles of the invention determines an adverse reaction to, or overdose of, a psychotropic medication. In a further example, a monitor determines the presence and degree of inebriation or intoxication. Still further alternative embodiments includes a monitor that detects a sudden fall by the wearer or an impact likely to cause bodily trauma such as a ballistic impact, being struck by a vehicle or other object, or an explosion in the proximity of the wearer. Still further alternative embodiments include a monitor to determine an acute medical crisis such has a heart attack, stroke or seizure. In one alternative arrangement, the monitor is able to detect a panic attack or other acute anxiety episode. In a further alternative arrangement, the monitor is able to determine from for example unsteady gait or reduced activity that there is frailty, illness or risk of medical crisis. In another alternative embodiment of the invention, the monitor is capable of detecting hazards to which the wearer has been exposed such as biological pathogens, neurotoxins, radiation, harmful chemicals or toxic environmental hazards.

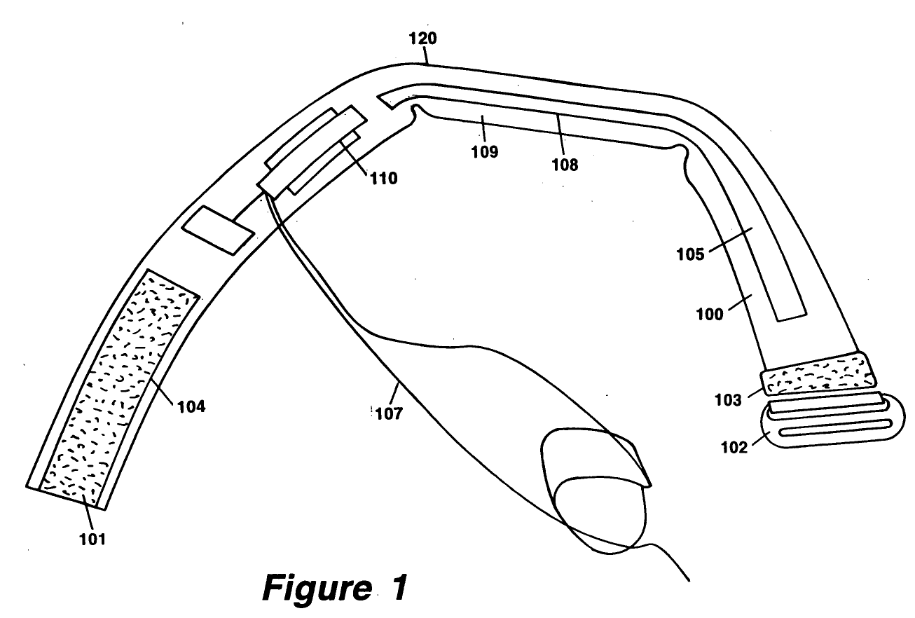

FIG. 1 is a picture of a chest strap holding sensors according to the present invention. The chest strap 120 holds sensors securely in proximity to the torso of a person (not shown). Sturdy cloth 100 forms the backbone of the chest strap 120, with soft high-friction cloth 105 placed on the inside to contact the skin of the torso so that the chest strap is optimally held in position. Should this not be sufficient, shoulder straps (not shown) can be attached to provide over-the-shoulder support. The chest strap 120 is cinched to appropriate tightness using a buckle 102 through which the opposite end 101 of the chest strap is fed.

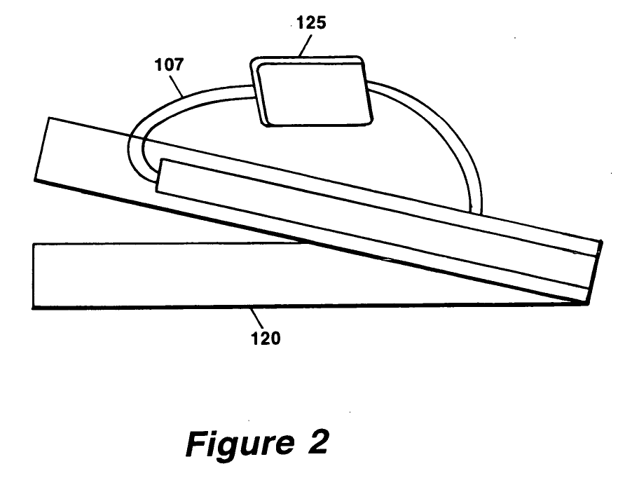

The hooks 103 and eyes 104 of Velcro complete the secure, non-moveable linkage. Wires 107 are used to link one or more sensors in the chest strap 120 to a hub 125, as shown in FIG. 2. The wires 107 emerge from conduits in the chest strap 120 leading from pockets or other topological features that hold or otherwise constrain the position of the sensors. Elastic cloth 109 with a spring constant much less than that of a piezo-electric strap 108 provides surrounding surface and structural strength as well as consistent look and feel for that part of the strap. The piezo-electric strap 108 increases and decreases voltage as it is stretched by the user's breathing out and in, thus provides a signal that can be used to determine whether the user is breathing, and if so, certain of the characteristics of that breathing. A pocket 110 holds a Polar Heart Monitor or other R-wave detector or other non-obtrusive heart beat detector, which communicates detailed information about heart beats wirelessly or by wire to the hub 125 (shown in FIG. 2), which is attached by Velcro or by other means to the outside of the chest strap, or to another on-body location. Alternative embodiments of the invention use radio communications to connect the sensors in the chest strap 120 to the hub 125 and so do not require the wires 107.

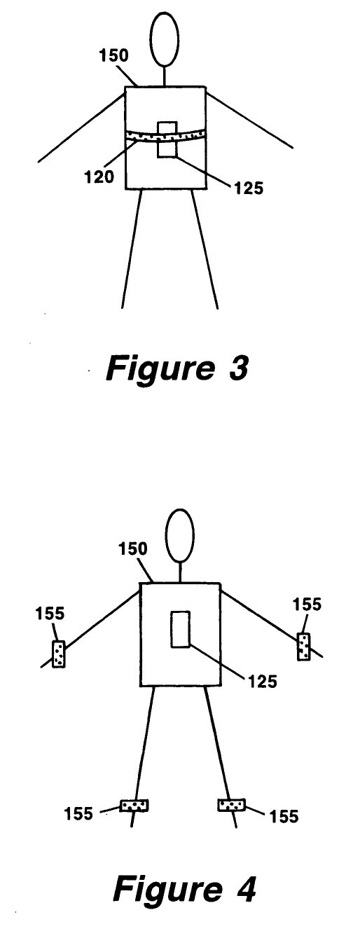

FIG. 3 is a block diagram of a first configuration of the hub and sensor placement on a human figure representation 150 according to principles of the invention. The human figure representation 150 is shown wearing a chest strap 120 having sensors (not shown) and a hub 125. The sensors include, for example, a piezoelectric breathing sensor and a polar heart monitor. The hub 125 includes, for example, an accelerometer and analytics. This example configuration of sensors can be used to monitor a patient with Parkinson's disease where pulmonary data, cardiovascular data and motion data are of interest.

FIG. 4 is a block diagram of a second configuration of the hub and sensor placement on a human figure representation 150 according to principles of the invention. The human figure representation 150 is shown wearing a hub 125 at the torso and sensors 155 at the wrists and ankles. The hub 125 includes, for example, an accelerometer and a wireless personal area network. The sensors are, for example, accelerometers and may include analytics. The sensors communicate wirelessly with the hub 125 through the wireless personal area network.

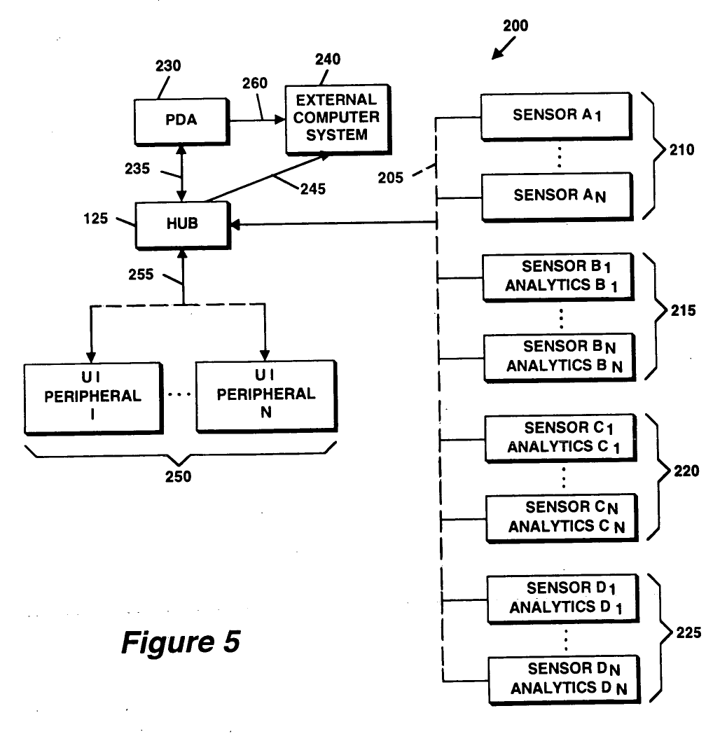

FIG. 5 is a block diagram of the hub and sensor network 200 according to the present invention. The hub and sensor network 200 includes a hub 125 connected through a first wired or a wireless personal area network (PAN) 205 a variety of sensors 210, 215, 220, 225. Sensors A 210 are without proactive communications abilities and instead are polled for data by the hub 125. Sensors B 215 are without proactive communications abilities however do include analytics. Sensors C 220 include both proactive communications and analytics. Sensors D 225 include proactive communications but are without analytics. The hub 125 is also connected to a PDA 230, or some other portable wireless communications device such as a cell phone, through a second wireless network 235. The hub 125 is further connected to an external local area network (LAN) or external computer system 240 through a wired or wireless connection 245. The hub 125 is still further connected to user interface peripherals 250 through a wired or wireless connection 255. The PDA 230 and external computer system 240 are connected through a wired or wireless connection 260.

In operation, the hub 125 communicates with and controls the sensors 210, 215, 220, 225, directing the sensors 210, 215, 220, 225 to collect data and to transmit the collected data to the hub 125. Those sensors 220, 225 with proactive communications send collected data to the hub 125 under preselected conditions. The hub 125 also communicates with and controls the user interface peripherals 250. The hub 125 further communicates with portable devices such as the PDA 230 and with external network or computer systems 240. The hub 125 communicates data and data analysis to the peripherals 250, portable devices 230 and external systems 240.

The hub and sensor network 200 shown here is merely an example network. Alternative embodiments of the invention include a network 200 with fewer types of sensors, for example, including a network 200 with only one type of sensor. Further alternative embodiments include a network 200 with a hub 125 connected to only a PDA 230. In still further alternative embodiments, the various devices in the network 200 are able to communicate with each other without using the hub as an intermediary device. In short, many types of hub, sensor, communications devices, computer devices and peripheral devices are possible within the scope of the present invention. The present invention is not limited to those combinations of devices listed here.

Sensor Hub Module with Internal Radio

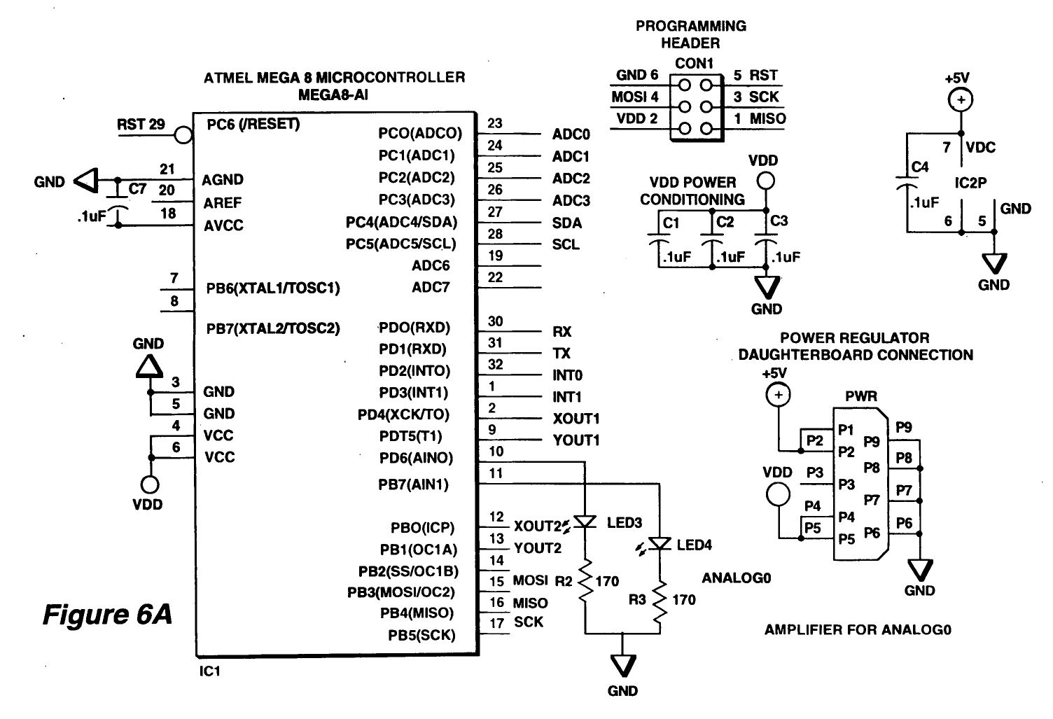

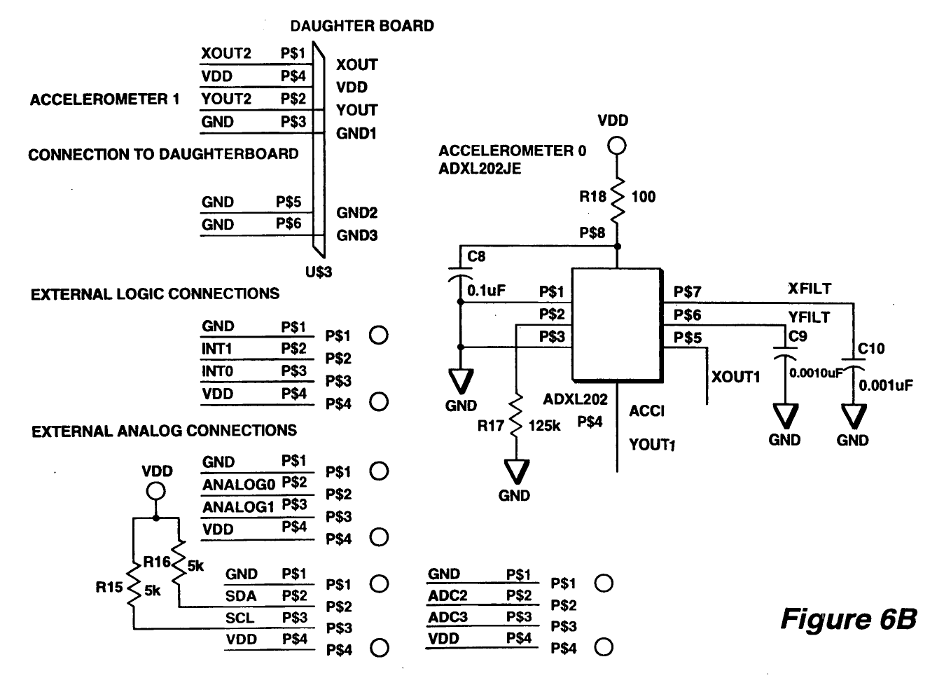

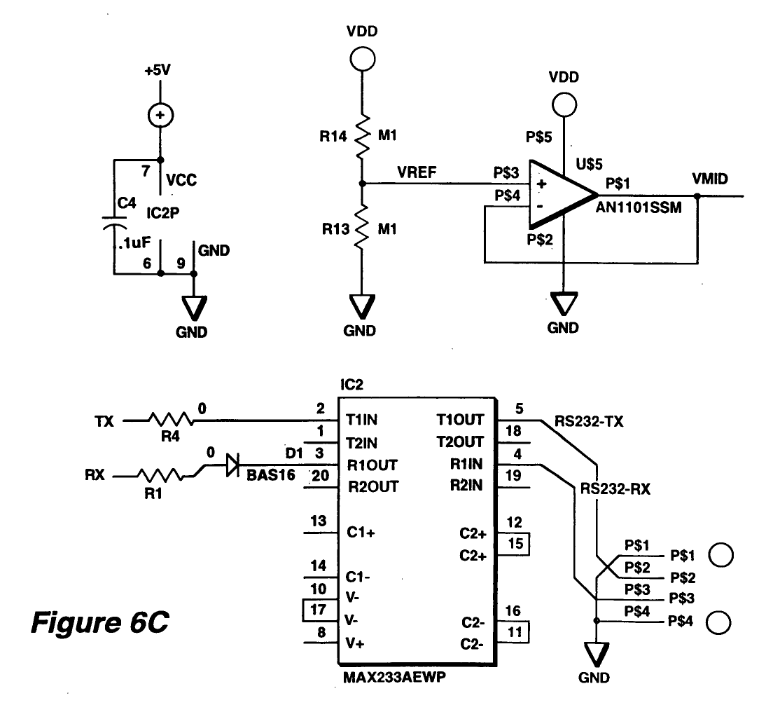

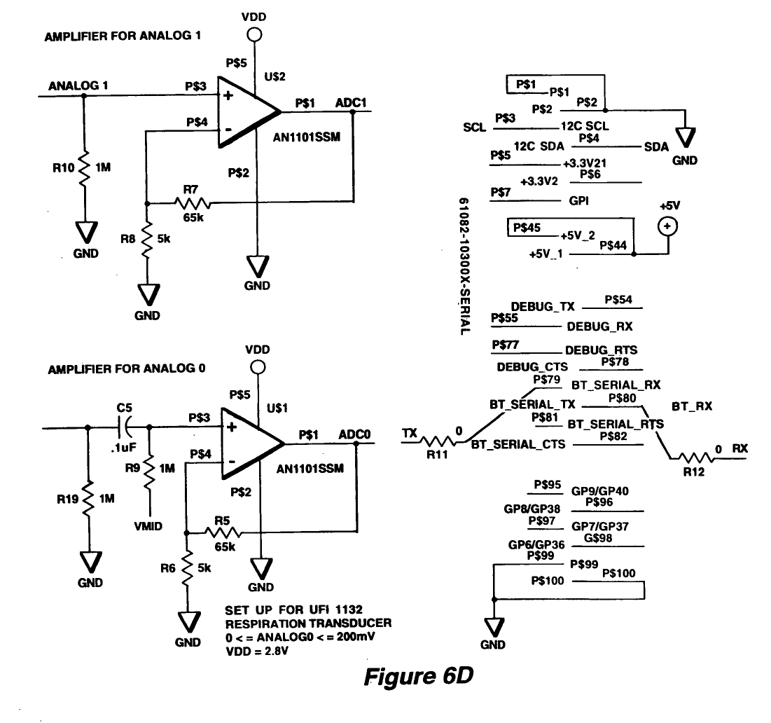

FIG. 6A, FIG. 6B, FIG. 6C and FIG. 6D together are a schematic diagram of a first sensor hub according to principles of the invention. FIG. 6A shows a first part of the first sensor hub, FIG. 6B shows a second part of the first sensor hub, FIG. 6C shows a third part of the first sensor hub and FIG. 6D shows a fourth part of the first sensor hub. The core of the sensor hub module in the preferred embodiment is an Atmel ATMega-8L micro-controller of Atmel Corporation of San Jose, Calif. The micro-controller is connected to two unbuffered analog inputs, two buffered analog inputs, two digital input/outputs, RS232, I2C, and two Analog Devices ADXL202E 2-axis accelerometers. One accelerometer is mounted flat on the sensor hub board, and the other is mounted perpendicular on a daughter board. This configuration allows for the detection of 3-axis acceleration.

The buffered analog inputs are composed of one AN1101SSM op-amp for each input. One of these op-amps is configured as a ground referenced DC amplifier, and the other is configured as a 1.65 Volt referenced AC amplifier. A third AN1101SSM provides a stable output for the 1.65 Volt reference.

The RS232 is routed to either the Cerfboard connector or to the Maxim MAX233AEWP RS232 line level shifter. This allows the sensor hub to be connected to the Cerfboard through the logic level serial or to other devices through RS232 level serial. The I2C bus is also routed through the Cerfboard connector to allow for alternative protocols to be used between the sensor hub and the Cerfboard.

All the devices except the RS232 line level shifter use the 3.3 Volt power rail. The line level shifter uses the 5 Volt power rail, and the 5 Volt power rail is also routed to the Cerfboard through its connector.

Power Module

The power module is composed of a Linear Technology LTC1143 dual voltage regulator, a Linear Technology LT1510-5 battery charger, and related passive components for both devices. The LTC1143 provides a switching regulated 3.3 Volt output and a 5.0 Volt output for input voltages that vary from 6 Volts to 8.4 Volts when running from the battery or 12 Volts to 15 Volts when running off an external power supply. The LT1510-5 charges a 2-cell Li-Poly battery using a constant I-V curve at 1 Amp when a 12 Volt to 15 Volt external power supply is used.

Life Signs Telemonitor Low-Power 2.4 GHz

FIG. 7A, FIG. 7B, FIG. 7C and FIG. 7D together are a schematic diagram of a second sensor hub according to principles of the invention. FIG. 7A is a first portion of the hub, FIG. 7B is a second portion of the hub, FIG. 7C is a third portion of the hub and FIG. 7D is a fourth portion of the hub. This hub is designed to provide sensor information over a short range radio link. By using a simple short range radio, the protocol can be handled on a lower power microcontroller. This reduces the space and power requirements from the 802.11 embodiment by not requiring a single board computer. The low power telemonitor is a single unit of hardware constructed from three modules.

The first module provides the power regulation system which outputs a 3.3 Volt power rail. The module can also optionally support a 5.0 Volt power rail and battery charger. The modules can run off of a Li-Poly 2-cell battery or a 12 volt regulated power source. These power rails are capable of handling loads of up to 450 mA. A power rail also charges the battery when an external power source is supplied. Due to the lower power requirements of this system, this module takes up less area and has shorter components than those used on the 802.11 system.

The second module contains the sensor hub and is nearly identical to the 802.11 version in terms of functionality. The difference is that the low power version provides its data via I2C to the third module instead of via RS232 to the Cerfboard.

The third module contains the low power, short-range radio system. This module takes the sensor data from the sensor hub module over I2C and transmits it over a short range 2.4 GHz radio link. The module may also be configured as a receiver for the sensor data transmissions, transferring the data to the destination data collection system over RS232 or I2C.

Sensor Hub Module

The core of the sensor hub module is an Atmel ATMega-8L micro-controller. The micro-controller is connected to two unbuffered analog inputs, two buffered analog inputs, two digital input/outputs, RS232, I2C, and two Analog Devices ADXL202E 2-axis accelerometers. One accelerometer is mounted flat on the sensor hub board, and the other is mounted perpendicular on a daughter board. This configuration allows for the detection of 3-axis acceleration.

The buffered analog inputs are composed of one AN1101SSM op-amp for each input. One of these op-amps is configured as a ground referenced DC amplifier, and the other is configured as a 1.65 Volt referenced AC amplifier. A third AN1101SSM provides a stable output for the 1.65 Volt reference.

The RS232 is routed to both a logic level connector or to the TI MAX3221CUE RS232 line level shifter. This allows the sensor hub to be connected to other devices through the logic level serial or RS232 level serial. The I2C bus is connected to the adjacent modules to handle the routing of sensor data between modules.

Radio Module

The radio module is composed of an Atmel ATMega-8L micro-controller and a Nordic VLSI nRF2401 2.4 GHz transceiver. The nRF2401 provides a 2.4 Ghz 1 Mbit short range wireless RF link. The micro-controller configures and handles all communications between the nRF2401 and the rest of the system.

The micro-controller has an I2C connection to the adjacent modules to allow it to transport sensor data to and from other modules on the system. It also connects to a TI MAX3221CUE RS232 line level shifter to allow the radio module to operate as a radio transceiver for an external device such as a laptop or PDA.

These modules contains all the needed passive components for the nRF2401 to operate in 1 Mbit mode including a PCB etched quarter wave antenna.

Power Module

The power modules contains 2 Maxim MAX750A switching power regulators, a Linear Technology LT1510-5 switching battery charger, and related passive components for each device. One MAX750A is configured to output a 3.3 Volt power rail, and the other is configured to output a 5.0 Volt power rail. Each of these rails is limited to 450 mA of current load. The input voltages to these regulators vary from 6 Volts to 8.4 Volts when running from the battery or is 12 Volts when running from an external regulated power supply. The LT1510-5 charges a 2-cell Li-Poly battery using a constant I-V curve at 1 Amp when a 12 Volt regulated external power supply is used.

FFT and Classifier Module

The Fast Fourier Transform (“FFT”) software is programmed in machine language on the Atmel processor. Because the Atmel computational capabilities are limited, the volume of data to be transformed substantially in real time is considerable, the FFT algorithm needs to run very fast. An algorithm using floating point is not generally compatible with present Atmel technology because floating point algorithms run too slow. Transforming the algorithm into fixed point made it possible for the algorithm to run with sufficient speed and with acceptable use of microcontroller resources.

Sensor information is input to the FFT algorithm, which computes the Fourier Transform as output. Such transformation of the original data into the frequency domain aids data analysis particularly in cases in which the phenomena are fundamentally oscillatory. Examples of such oscillatory data are ambulatory motion, heart beat, breathing, and motion on a vehicle that is traveling. This output is then input to a Classifier module, which analyzes and recognizes the pattern or patterns inherent in the data and compares them to patterns it has been trained to recognize using a statistical algorithm. The Classifier module output consists of one or more matched patterns along with the confidence level for the match.

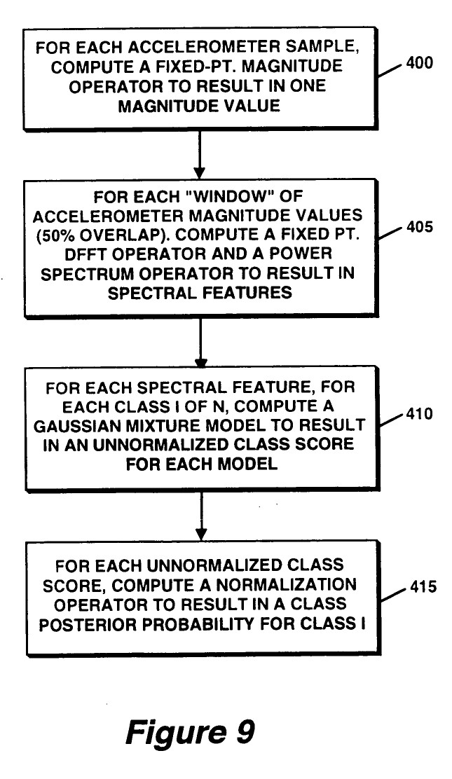

FIG. 9 is a flow chart of the process of the Classifier module.

At step 400, the Classifier module executes the following:

For each accelerometer sample, do:

three axis accelerometer sample→{fixed-point magnitude operator}
→one magnitude value
   At step 405, the Classifier module executes the following:
   For each “window” of, for example, 64 accelerometer magnitude values (50% overlap), do:

64 magnitude values→{fixed point DFFT operator}
→{power spectrum (mag square) operator}
→thirty one spectral features.

Sample numbers are typically any power of two. If a larger number of values is used, more memory is generally required.

At step 410, the Classifier module executes the following:

For each vector of 31 spectral features, do:

for each class (Gaussian mixture model) i of n, do:
31 spectral features→{Gaussian mixture model i}
   →sᵢ(class score for model i)
      Result is n unnormalized class scores.
      At step 415, the Classifier module executes the following:
      For each unnormalized sᵢ, do:

sᵢ{normalization operator}
→pᵢ(class posterior probability for class i)
   Result is class posterior probabilities for each class, given the window of 31 spectral features.

The display of the output information in the presently preferred embodiment is a listing of patterns matched along with confidence levels. Those skilled in the art will recognize that many alternative displays can be useful. Examples of such displays include a red-yellow-green light for each of one or more matches, and a color coded thermometer with the color representing an action to be taken and the height of the indicator a measure of the confidence with which the Classifier determined this to derive from a correct data-model match.

The manner in which the information is visualized is supportive of the core feature of “alarming” based on the output of the classifier. The core feature of the “proactive telemonitor” is that it is proactive. In some embodiments of the invention, nothing is displayed until the health state classifier (or environmental conditions classifier, the injury classifier, etc.) detects that there is a problem, and calls for help. This implementation is feasible because it utilizes principled classification to drive proactive communications and user interaction rather than merely displaying information or sending an alarm upon the overly simplistic criterion of some data parameter being exceeded.

In alternative embodiments of the present invention, other types of microcontrollers other than the Atmel microprocessor may be used. Many low complexity, basic microprocessors are suitable for use in the present invention. The present invention is not limited to the microprocessors listed here.

It is to be understood that the above-identified embodiments are simply illustrative of the principles of the invention. Various and other modifications and changes may be made by those skilled in the art which will embody the principles of the invention and fall within the spirit and scope thereof.

We claim:

1. A wearable hub for a remote monitor device, the hub positioned on the body of a person, comprising:
a data receiver to receive transmitted data from at least one sensor positioned on the person;
an analysis device to take the received data as input, the analysis device to determine a condition of the person in response to the data; and
a transmitter to transmit the condition to an external device.

2. The wearable hub of claim 1 wherein the analysis device further comprises a data classifier, the data classifier employing statistical classification techniques to determine the condition from the received data.

3. The wearable hub of claim 2 wherein the patterns of data are organized into data classes and the data classifier determines the condition according to which data class the received data belongs.

4. The wearable hub of claim 1 wherein the analysis device further comprises a model analysis system, the model analysis system storing model data and associated rules, the model analysis system to determine the condition by applying the model data and associated rules to the received data.

5. The wearable hub of claim 1 wherein the external device is an external communications device.

6. The wearable hub of claim 5 wherein the external communications device is a cellular telephone.

7. The wearable hub of claim 1 wherein the external device is a personal digital assistant.

8. The wearable hub of claim 1 wherein the external device is an external display device.

9. The wearable hub of claim 1 wherein the external device is an interface device able to transmit data back to the hub in response to transmissions from the hub.

10. The wearable hub of claim 1 wherein the external device is an external network able to connect to a plurality of external devices.

11. The wearable hub of claim 1 wherein the external device is an external computer device.

12. An on-body monitoring network, comprising:
a plurality of sensors placed on a person's body;
at least one analytic device to analyze data from at least one of the plurality of sensors, the analytic device to determine a condition of the person; and
a hub to control the plurality of sensors and the at least one analytic device, the hub to communicate the condition to at least one external device.

13. The on-body monitoring network of claim 12 wherein the condition determined by the analytic device is whether the person is alive or dead.

14. The on-body monitoring device of claim 12 wherein the condition determined by the analytic device is a medication detection of a Parkinson's disease patient.

15. The on-body monitoring device of claim 12 wherein the condition determined by the analytic device is a degree of progression of Parkinson's disease.

16. The on-body monitoring device of claim 12 wherein the condition determined by the analytic device is the progression or diagnosis of a neurological condition that effects patient motion.

17. The on-body monitoring device of claim 12 wherein the condition determined by the analytic device is adverse reaction a medication.

18. The on-body monitoring device of claim 12 wherein the condition determined by the analytic device is a degree of intoxication.

19. The on-body monitoring device of claim 12 wherein the condition determined by the analytic device is a sudden fall.

20. The on-body monitoring device of claim 12 wherein the condition determined by the analytic device is an acute medical crisis.

21. The on-body monitoring device of claim 12 wherein the condition determined by the analytic device is a panic attack.

22. The on-body monitoring device of claim 12 wherein the condition determined by the analytic device is an impact likely to cause bodily trauma.

23. The on-body monitoring device of claim 12 wherein the condition determined by the analytic device is unsteady gait.

24. The on-body monitoring device of claim 12 wherein the condition determined by the analytic device is related to the performance of a physically demanding activity.

25. The on-body monitoring device of claim 12 wherein the condition determined by the analytic device is exposure to biological pathogens.

26. The on-body monitoring device of claim 12 wherein the condition determined by the analytic device is exposure to a toxic hazard.

27. The on-body monitoring network of claim 12 wherein the analytic device further comprises a subsystem that determines a level of confidence in the determined condition.

28. The on-body monitoring network of claim 12 wherein the at least one external device is an external computer device including a display.

29. The on-body monitoring network of claim 28 wherein the display is capable of displaying real-time data curves.

30. The on-body monitoring network of claim 28 wherein the display is capable of visualizing data in human-intelligible form.

31. The on-body monitoring network of claim 12 wherein the at least one external device is a personal digital assistant.

32. The on-body monitoring network of claim 12 wherein the at least one external device is a laptop computer.

33. The on-body monitoring network of claim 12 further comprising a chest strap wherein the plurality of sensors are located on the chest strap, and wherein the hub is located on the chest strap.

34. The on-body monitoring network of claim 12 wherein the plurality of sensors are distributed at various locations about the person's body and wherein the plurality of sensors, the analytic device and the hub communicate wirelessly.

35. The on-body monitoring network of claim 34 wherein the at least one analytic device is associated with one of the plurality of distributed sensors.

36. The on-body monitoring network of claim 34 further comprising a plurality of analytics wherein each analytic is associated with one of the plurality of sensors thereby forming a distributed analytic network.

37. The on-body monitoring network of claim 36 wherein one of the plurality of analytics is associated with the hub, the analytic in the hub receives input from the analytics associated with each of the sensors.

38. The on-body monitoring network of claim 12 wherein the at least one analytic device includes a processor capable of performing Fast Fourier Transform calculations.

39. The on-body monitoring network of claim 38 wherein the processor a low power microcontroller.

40. The on-body monitoring network of claim 38 wherein the hub further comprises a data classifier implementing a real-time statistical classification system, the data classifier to determine the condition from model features.

41. A remote monitoring device, comprising:
a plurality of on-body sensors;
an on-body hub receiving data input from the plurality of on-body sensors, the on-body hub to determine a condition from the received data, the hub to communicate the condition to an external device; and
a chest strap holding the on-body sensors and the on-body hub in place.

42. The remote monitoring device of claim 41 wherein the chest strap further comprises at least one shoulder strap to hold the chest strap.
